(19) United States
(12) Patent Application Publication
(10) Pub. No.: US 2022/0228209 A1
(43) Pub. Date: Jul. 21, 2022

(54) DNA METHYLATION SEQUENCING ANALYSIS METHODS

(71) Applicants: Genecast Biotechnology Co., Ltd., Wuxi City (CN); Genecast (Beijing) Biotechnology Co., Ltd., Beijing (CN); Genecast (Wuxi) Precision Medicine Diagnostic Laboratory Co., Ltd., Wuxi City (CN)

(72) Inventors: Tiancheng Han, Beijing (CN); Xiaofeng Song, Beijing (CN); Jianing Yu, Beijing (CN); Yuanyuan Hong, Beijing (CN); Weizhi Chen, Beijing (CN); Bo Du, Beijing (CN)

(73) Assignees: Genecast Biotechnology Co., Ltd., Wuxi City (CN); Genecast (Beijing) Biotechnology Co., Ltd., Beijing (CN); Genecast (Wuxi) Precision Medicine Diagnostic Laboratory Co., Ltd., Wuxi City (CN)

(21) Appl. No.: 17/490,549

(22) Filed: Sep. 30, 2021

Related U.S. Application Data
(63) Continuation of application No. PCT/CN2021/091761, filed on Apr. 30, 2021.

(30) Foreign Application Priority Data
Jan. 20, 2021 (CN) 202110072090.5
Jan. 21, 2021 (CN) 202110078570.2

(51) Int. Cl. C12Q 1/6874; G16B 20/20; G16B 40/00; C12Q 1/6886

(52) U.S. Cl. CPC C12Q 1/6874; C12Q 1/6886; G16B 40/00; G16B 20/20

(57) ABSTRACT

Embodiments of the invention provides methods for determining a methylation score of DNA and determining a ctDNA Fraction (CTDF) value. Additional embodiments are as described herein.

### BACKGROUND OF THE INVENTION

DNA methylation is an epigenetic mechanism that occurs due to the addition of a methyl group to DNA, thereby modifying the function of genes and affecting gene expression. In some genomic regions, the methylation statuses of neighboring CpG sites are highly correlated. As a result, the methylation status of a single fragment on these sites is usually consistent with the neighboring sites.

In bisulfite or enzymatic conversion of DNA, only unmethylated cytosine (C) is converted into uracil (U). After PCR amplification of converted DNA, the unmethylated Cs of the template fragments become thymines (Ts) in the amplified DNA, and the methylated Cs of the template fragments remain as Cs in the amplified DNA. Thus, the methylation status of CpG sites can be distinguished by conversion and amplification.

Methylation levels can be measured as a ratio called the beta-value, which is the number of Cs divided by the number of Cs plus Ts on this site. Ideally, if the unmethylated Cs are completely converted, the beta-value would be precisely calculated. However, the conversion rate is not 100%, and there also may be sequencing errors during amplification. Typically, then, beta-values are biased, which then leads to biased predictions based on the beta-values.

Cell-free DNA (cfDNA) comprises highly degraded DNA fragments, which are detectable in the peripheral blood of every human. In healthy individuals, the vast majority of cfDNA is derived from the hematopoietic system. In cancer patients, cfDNA includes circulating tumor DNA (ctDNA) shed from tumor cells into the circulation. These fragments retain cancer-specific marks from the originating cancer cells. Therefore, analyses of cfDNA can be used to diagnose cancer at an early stage or monitor minimal residual disease.

However, for ctDNA cancer models, the bias of beta-values significantly interferes with analysis since the ctDNA fraction (CTDF) of plasma/body fluid is low, and beta-values can be greatly distorted by noise. Bias of beta-values also impacts analysis of DNA samples from body fluids and tissues.

Thus, there is a need for better methods to analyze DNA with less bias.

### BRIEF SUMMARY OF THE INVENTION

In embodiments, the invention provides a method for determining a methylation score of DNA, the method comprising: (a) providing a sample from a subject; (b) isolating DNA from the sample of (a); (c) treating isolated DNA of (b) with bisulfate or enzyme to perform conversion of unmethylated cytosines in the DNA; (d) performing library construction of the converted DNA of (c) by paired end next generation sequencing (NGS); (e) obtaining sequencing data from the paired end NGS of (d) and determining DNA sequences of DNA fragments present, wherein the sequence of a DNA fragment is determined by merging the sequences of read-pairs for the DNA fragment; (f) identifying methylation status of DNA fragments of (e) by comparing a reference genome to the sequencing data of (e) to determine if a cytosine base in a CpG site within a DNA fragment is methylated or unmethylated; (g) calculating for Methylation-Correlated Blocks (MCBs) a Methylated Fragment Ratio (MFR) value or an Unmethylated Fragment Ratio (UFR) value or both a MFR value and an UFR value, wherein the MCBs are based on CpGs pre-determined within the DNA; and (h) calculating a p-value for each of selected differential MCBs, wherein the selected differential MCBs are selected based on pre-determined MFR and UFR values, and wherein the p-value is based on a pre-determined baseline distribution of MFR values if selected differential MCBs are hypermethylated or UFR values if selected differential MCBs are hypomethylated; and (i) calculating a methylation score using the equation

$${Score}_{n} = \frac{- 2 {\sum }_{j = 1}^{J} {c}_{j} \cdot ln {p}_{n , j}}{{\sum }_{j = 1}^{J} {c}_{j}}$$

wherein cⱼ is the weight of MCBⱼ within sampleₙ, p_{n,j} is the p-value of (h) for MCBⱼ in sampleₙ, wherein sampleₙ, has J number of MCB, and wherein the methylation score is a hypermethylation score if selected differential MCBs in (h) are hypermethylated and is a hypomethylation score if selected differential MCBs in (h) are hypomethylated and is a hybrid methylation score if selected differential MCBs in (h) comprise both hypermethylated and hypomethylated MCBs.

In embodiments, the invention provides a method for determining a ctDNA Fraction (CTDF) value, the method comprising: (a) providing a sample from a subject; (b) isolating DNA from the sample of (a); (c) treating isolated DNA of (b) with bisulfate or enzyme to perform conversion of unmethylated cytosines in the DNA; (d) performing library construction of the converted DNA of (c) by paired end next generation sequencing (NGS); (e) obtaining sequencing data from the paired end NGS of (d) and determining DNA sequences of DNA fragments present, wherein the sequence of a DNA fragment is determined by merging the sequences of read-pairs for the DNA fragment; (f) identifying methylation status of DNA fragments of (e) by comparing a reference genome to the sequencing data of (e) to determine if a cytosine base in a CpG site within a DNA fragment is methylated or unmethylated; (g) calculating for Methylation-Correlated Blocks (MCBs) a Methylated Fragment Ratio (MFR) value, wherein the MCBs are based on CpGs pre-determined within the DNA; and (h) calculating tumor and non-tumor likelihood values for each of selected differential MCBs, wherein the selected differential MCBs are selected based on pre-determined MFR values, and wherein the tumor and non-tumor likelihood values are based on a pre-determined beta distribution of MFR values calculated in (g); and (i) calculating a ctDNA Fraction (CTDF) value based on the tumor and non-tumor likelihood values determined in (h) using the equation

$$log P ( F \vert θ , M ) = {\sum }_{c} {w}_{c} \cdot log ( θ \cdot P ( {f}^{c} \vert {m}_{j}^{T} ) + ( 1 - θ ) \cdot P ( {f}^{c} \vert {m}_{j}^{N} ) )$$

wherein j is the MCB covered by f^{c}; P(f^{c}|m^{T}ⱼ) and P(f^{c}|m^{N}ⱼ) are

$$P ( f \vert {m}_{j}^{T} ) = {\prod }_{h} \frac{B ( {f}_{h} + {α}_{j}^{T} , 1 - {f}_{h} + {β}_{j}^{T} )}{B ( {α}_{j}^{T} , {β}_{j}^{T} )}$$

$$and$$

$$P ( f \vert {m}_{j}^{N} ) = {\prod }_{h} \frac{B ( {f}_{h} + {α}_{j}^{N} , 1 - {f}_{h} + {β}_{j}^{N} )}{B ( {α}_{j}^{N} , {β}_{j}^{N} )}$$

respectively, for a given fragment f^{c}, α^{T}ⱼ, β^{T}ⱼ, α^{N}ⱼ and β^{N}ⱼ are parameters of tumor or normal class beta distributions of MFR on MCB j, which is estimated from m^{T}ⱼ and m^{N}ⱼ; m^{T}ⱼ is the tumor class methylation pattern on MCBj and m^{N}ⱼ is the normal class methylation pattern on MCBj; fₕ is 0 or 1, representing methylated or unmethylated status of the CpG site h in fragment f; θ is the CTDF estimated by a grid search; and w_{c} is the weight assigned for f^{c}.

Additional embodiments are as described herein.

### BRIEF DESCRIPTION OF THE DRAWINGS

FIGS. 1 and 2 each present graphs for colorectal cancer (left) and lung cancer (right) plotting sensitivity (%) vs. specificity (%) for comparing models as described in Example 8. FIG. 1 presents comparisons of models of methylation score. FIG. 2 presents comparisons of models of CancerDetector.

### DETAILED DESCRIPTION OF THE INVENTION

It has been surprisingly and unexpectedly discovered that the methods as described herein improve the performance of epigenetic models. Without wishing to be bound by any theory, the methods as described herein, e.g., suppress noise due to incomplete conversion or sequencing errors by discriminating and removing unreliable reads/fragments and take advantage of the correlation between CpG sites by identifying the true methylation status of a CpG site based on the status of itself and the statuses of its neighboring sites.

In embodiments, the invention provides a method for determining a methylation score of DNA, the method comprising: (a) providing a sample from a subject; (b) isolating DNA from the sample of (a); (c) treating isolated DNA of (b) with bisulfate or enzyme to perform conversion of unmethylated cytosines in the DNA; (d) performing library construction of the converted DNA of (c) by paired end next generation sequencing (NGS); (e) obtaining sequencing data from the paired end NGS of (d) and determining DNA sequences of DNA fragments present, wherein the sequence of a DNA fragment is determined by merging the sequences of read-pairs for the DNA fragment; (f) identifying methylation status of DNA fragments of (e) by comparing a reference genome to the sequencing data of (e) to determine if a cytosine base in a CpG site within a DNA fragment is methylated or unmethylated; (g) calculating for Methylation-Correlated Blocks (MCBs) a Methylated Fragment Ratio (MFR) value or an Unmethylated Fragment Ratio (UFR) value or both a MFR value and an UFR value, wherein the MCBs are based on CpGs pre-determined within the DNA; and (h) calculating a p-value for each of selected differential MCBs, wherein the selected differential MCBs are selected based on pre-determined MFR and UFR values, and wherein the p-value is based on a pre-determined baseline distribution of MFR values if selected differential MCBs are hypermethylated or UFR values if selected differential MCBs are hypomethylated; and (i) calculating a methylation score using the equation

$${Score}_{n} = \frac{- 2 {\sum }_{j = 1}^{J} {c}_{j} \cdot ln {p}_{n , j}}{{\sum }_{j = 1}^{J} {c}_{j}}$$

wherein cⱼ is the weight of MCBⱼ within sampleₙ, p_{n,j} is the p-value of (h) for MCBⱼ in sampleₙ, wherein sampleₙ, has J number of MCB, and wherein the methylation score is a hypermethylation score if selected differential MCBs in (h) are hypermethylated and is a hypomethylation score if selected differential MCBs in (h) are hypomethylated and is a hybrid methylation score if selected differential MCBs in (h) comprise both hypermethylated and hypomethylated MCBs.

Bisulfite and enzymatic conversion of DNA for sequencing purposes are well known in the art, and any suitable method may be used. An exemplary method of enzymatic conversion is enzymatic methyl-seq, e.g., commercially available from New England Biolabs as NEBNext® Enzymatic Methyl-Seq (Ipswich, Mass., USA).

In embodiments, the selected differential MCBs in (h) are hypermethylated and wherein a hypermethylation score is calculated in (i). In embodiments, the selected differential MCBs in (h) are hypomethylated wherein a hypomethylation score is calculated in (i). In embodiments, the selected differential MCBs in (h) comprise both hypermethylated and hypomethylated MCBs and wherein a hybrid methylation score is calculated in (i).

In embodiments, the cⱼ is equal to count_{n,j}, or −count_{n,j}·ln FDRⱼ, wherein count_{n,j} is the number of fragments from sampleₙ on MCBⱼ, and FDAⱼ is false discovery/positive rate of MCBⱼ. Additional exemplary weights are described within the Examples.

In embodiments, the sample is a plasma sample. In embodiments, the DNA is cell-free DNA.

In embodiments, the invention provides a method of treating a subject having cancer, the method comprising: (A) determining the methylation score of DNA of a test subject according to a method described above; and (B) determining that the test subject has cancer based on the methylation score of (A); and (C) treating the test subject.

Without wishing to be bound by any theory, generally, the methylation score is associated with the fraction of ctDNA fragments in the plasma, when ctDNA is detected. ctDNA fraction in late stage cancer and some specific cancer types tend to be higher, which leads to a higher methylation score.

In embodiments, the invention provides a method for determining a ctDNA Fraction (CTDF) value, the method comprising: (a) providing a sample from a subject; (b) isolating DNA from the sample of (a); (c) treating isolated DNA of (b) with bisulfate or enzyme to perform conversion of unmethylated cytosines in the DNA; (d) performing library construction of the converted DNA of (c) by paired end next generation sequencing (NGS); (e) obtaining sequencing data from the paired end NGS of (d) and determining DNA sequences of DNA fragments present, wherein the sequence of a DNA fragment is determined by merging the sequences of read-pairs for the DNA fragment; (f) identifying methylation status of DNA fragments of (e) by comparing a reference genome to the sequencing data of (e) to determine if a cytosine base in a CpG site within a DNA fragment is methylated or unmethylated; (g) calculating for Methylation-Correlated Blocks (MCBs) a Methylated Fragment Ratio (MFR) value, wherein the MCBs are based on CpGs pre-determined within the DNA; and (h) calculating tumor and non-tumor likelihood values for each of selected differential MCBs, wherein the selected differential MCBs are selected based on pre-determined MFR values, and wherein the tumor and non-tumor likelihood values are based on a pre-determined beta distribution of MFR values calculated in (g); and (i) calculating a ctDNA Fraction (CTDF) value based on the tumor and non-tumor likelihood values determined in (h) using the equation

$$log P ( F \vert θ , M ) = {\sum }_{c} {w}_{c} \cdot log ( θ \cdot P ( {f}^{c} \vert {m}_{j}^{T} ) + ( 1 - θ ) \cdot P ( {f}^{c} \vert {m}_{j}^{N} ) )$$

wherein j is the MCB covered by f^{c}; P(f^{c}|m^{T}ⱼ) and P(f_{c}|m^{N}ⱼ) are

$$P ( f \vert {m}_{j}^{T} ) = {\prod }_{h} \frac{B ( {f}_{h} + {α}_{j}^{T} , 1 - {f}_{h} + {β}_{j}^{T} )}{B ( {α}_{j}^{T} , {β}_{j}^{T} )}$$

$$and$$

$$P ( f \vert {m}_{j}^{N} ) = {\prod }_{h} \frac{B ( {f}_{h} + {α}_{j}^{N} , 1 - {f}_{h} + {β}_{j}^{N} )}{B ( {α}_{j}^{N} , {β}_{j}^{N} )}$$

respectively, for a given fragment f^{c}; α^{T}ⱼ, β^{T}ⱼ, α^{N}ⱼ and β^{N}ⱼ are parameters of tumor or normal class beta distributions of MFR on MCB j, which is estimated from m^{T}ⱼ and m^{N}ⱼ; m^{T}ⱼ is the tumor class methylation pattern on MCBj and m^{N}ⱼ is the normal class methylation pattern on MCBj; fₕ is 0 or 1; θ is estimated by a grid search; and w_{c} is the weight assigned for f^{c}.

In embodiments, w_{c} is one of

MR
MR²
\sqrt{MR}
$\frac{1}{log MR}$
$\{ \begin{matrix}1 , & MR \geq {MR}_{b} \vert MR \leq {MR}_{a} \\ 0 , & {MR}_{a} < MR < {MR}_{b}\end{matrix}$

wherein MR is the percentage of methylated CpGs of each fragment, MR_{b} is the threshold of MR for methylated fragments, and MRₐ is the threshold of MR for unmethylated fragments.

In embodiments, the sample is a plasma sample. In embodiments, the DNA is cell-free DNA.

In embodiments, the invention provides a method of treating a subject having cancer, the method comprising: (A) determining the ctDNA Fraction (CTDF) value of a test subject according to a method described above; and (B) determining that the test subject has cancer based on the CTDF of (A); and (C) treating the test subject.

For the inventive methods, pre-determination, as described herein, is based on knowledge of DNA prior to performing the inventive methods and/or is based on knowledge of the results of previously performing the inventive methods on other subjects. The other subjects may be healthy subjects, and the performance of the inventive methods may be to establish baseline values, threshold values, and/or distributions against which to compare future-determined values. For example, MFR and/or UFR values may be established for MCBs of healthy subjects such that certain of the MCBs are deemed differential MCBs based on those values. Also, for example, a distribution of MFR or UFR values may be established for subjects against which another such value may be compared to determine a p-value associated with the value. The Examples provide such exemplary methods.

Methylation scores and CTDF values are each higher in subjects with cancer compared to subjects without cancer. A threshold can be set which depends on the score/value distribution in healthy subjects. Subjects with scores/values higher than the threshold are predicted as diseased. Without prior knowledge of the score/value distribution among cases, the highest score/value in healthy subjects or the 95th percentile can be used as the threshold. The threshold controls the trade-off between sensitivity and specificity (or between positive and negative predictive values (PPV and NPV, respectively)). For example, if the method is applied to cancer diagnoses in high-risk populations, where higher sensitivities are desirable to minimize the number of false negatives, lower thresholds are preferable. Also for example, if the method is applied to screening tests for diseases that are not life-threatening such as prostate cancer, where higher PPVs are desirable to reduce the overtreatment caused by false positives, higher thresholds are preferable. If there is no preference for sensitivity/specificity/PPV/NPV, the optimal threshold can be obtained from a receiver operating characteristic curve (ROC curve). Each point on the curve gives the specificity (1−x) and the sensitivity (y) for a threshold value. The optimal threshold can be represented by either the point closest to the (0, 1) or the one that maximizes the distance from the diagonal line (y=x).

In embodiments, each of the inventive methods can be performed on other DNAs, where the samples used to build the baseline would be changed accordingly. For example, if tissue DNA is tested, baseline samples in the Methylation Score Model and normal baseline samples in the CancerDetector Model would be normal tissues. Thus, blood, biopsy, bodily fluid, and tissues may be samples from which DNA is obtained for the inventive methods.

Sequencing data may be obtained by any suitable next generation sequencing method, e.g., by direct sequencing (e.g., whole genome bisulfite sequencing, WGBS) or hybridization to a pre-designed probe panel to capture the target region for sequencing. Data can also be obtained by Reduced Representation Bisulfite-Sequencing (RRBS), a protocol that uses one or multiple restriction enzymes on the genomic DNA to enrich GC-rich sequence-specific fragmentation. It is more cost-effective than WGBS and covers about 4 million CpGs.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods can provide any amount or any level of treatment or prevention of cancer in a subject. Furthermore, the treatment or prevention provided by the method can include treatment or prevention of one or more conditions or symptoms of the disease being treated or prevented. Also, for purposes herein, “prevention” can encompass delaying the onset of the disease, or a symptom or condition thereof.

In embodiments, for any of the inventive methods the subject is a human.

The following includes certain aspects of the invention.

1. A method for determining a methylation score of DNA, the method comprising:

(a) providing a sample from a subject;

(b) isolating DNA from the sample of (a);

(c) treating isolated DNA of (b) with bisulfite or enzyme to perform conversion of unmethylated cytosines in the DNA;

(d) performing library construction of the converted DNA of (c) by paired end next generation sequencing (NGS);

(e) obtaining sequencing data from the paired end NGS of (d) and determining DNA sequences of DNA fragments present,

wherein the sequence of a DNA fragment is determined by merging the sequences of read-pairs for the DNA fragment;

(f) identifying methylation status of DNA fragments of (e) by comparing a reference genome to the sequencing data of (e) to determine if a cytosine base in a CpG site within a DNA fragment is methylated or unmethylated;

(g) calculating for Methylation-Correlated Blocks (MCBs) a Methylated Fragment Ratio (MFR) value or an Unmethylated Fragment Ratio (UFR) value or both a MFR value and an UFR value, wherein the MCBs are based on CpGs pre-determined within the DNA; and

(h) calculating a p-value for each of selected differential MCBs,

wherein the selected differential MCBs are selected based on pre-determined MFR and UFR values, and

wherein the p-value is based on a pre-determined baseline distribution of MFR values if selected differential MCBs are hypermethylated or UFR values if selected differential MCBs are hypomethylated; and

(i) calculating a methylation score using the equation

$${Score}_{n} = \frac{- 2 {\sum }_{j = 1}^{J} {c}_{j} \cdot ln {p}_{n , j}}{{\sum }_{j = 1}^{J} {c}_{j}}$$

wherein cⱼ is the weight of MCBⱼ within sampleₙ, p_{n,j} is the p-value of (h) for MCBⱼ in sampleₙ,

wherein sampleₙ, has J number of MCB, and

wherein the methylation score is a hypermethylation score if selected differential MCBs in (h) are hypermethylated and is a hypomethylation score if selected differential MCBs in (h) are hypomethylated and is a hybrid methylation score if selected differential MCBs in (h) comprise both hypermethylated and hypomethylated MCBs.

2. The method of aspect 1, wherein selected differential MCBs in (h) are hypermethylated and wherein a hypermethylation score is calculated in (i).

3. The method of aspect 1, wherein selected differential MCBs in (h) are hypomethylated wherein a hypomethylation score is calculated in (i).

4. The method of aspect 1, wherein selected differential MCBs in (h) comprise both hypermethylated and hypomethylated MCBs and wherein a hybrid methylation score is calculated in (i).

5. The method of any one of aspects 1-4, wherein the cⱼ is equal to

count_{n,j}, or

−count_{n,j}·ln FDRⱼ,

wherein count_{n,j} is the number of fragments from sampleₙ on MCBⱼ, and FDAⱼ is false discovery/positive rate of MCBⱼ.

6. The method of any one of aspects 1-5, wherein the sample is a plasma sample.

7. The method of aspect 6, wherein the DNA is cell-free DNA.

8. A method of treating a subject having cancer, the method comprising:

(A) determining the methylation score of DNA of a test subject according to the method of any one of aspects 1-7; and

(B) determining that the test subject has cancer based on the methylation score of (A); and

(C) treating the test subject.

9. A method for determining a ctDNA Fraction (CTDF) value, the method comprising:

(a) providing a sample from a subject;

(b) isolating DNA from the sample of (a);

(c) treating isolated DNA of (b) with bisulfite or enzyme to perform conversion of unmethylated cytosines in the DNA;

(d) performing library construction of the converted DNA of (c) by paired end next generation sequencing (NGS);

(e) obtaining sequencing data from the paired end NGS of (d) and determining DNA sequences of DNA fragments present,

wherein the sequence of a DNA fragment is determined by merging the sequences of read-pairs for the DNA fragment;

(f) identifying methylation status of DNA fragments of (e) by comparing a reference genome to the sequencing data of (e) to determine if a cytosine base in a CpG site within a DNA fragment is methylated or unmethylated;

(g) calculating for Methylation-Correlated Blocks (MCBs) a Methylated Fragment Ratio (MFR) value, wherein the MCBs are based on CpGs pre-determined within the DNA; and

(h) calculating tumor and non-tumor likelihood values for each of selected differential MCBs,

wherein the selected differential MCBs are selected based on pre-determined MFR values, and

wherein the tumor and non-tumor likelihood values are based on a pre-determined beta distribution of MFR values calculated in (g); and

(i) calculating a ctDNA Fraction (CTDF) value based on the tumor and non-tumor likelihood values determined in (h) using the equation

$$log P ( F \vert θ , M ) = {\sum }_{c} {w}_{c} \cdot log ( θ \cdot P ( {f}^{c} \vert {m}_{j}^{T} ) + ( 1 - θ ) \cdot P ( {f}^{c} \vert {m}_{j}^{N} ) )$$

wherein

j is the MCB covered by f^{c};

P(f^{c}|m^{T}ⱼ) and P(f^{c}|m^{N}ⱼ) are

$$P ( f \vert {m}_{j}^{T} ) = {\prod }_{h} \frac{B ( {f}_{h} + {α}_{j}^{T} , 1 - {f}_{h} + {β}_{j}^{T} )}{B ( {α}_{j}^{T} , {β}_{j}^{T} )}$$

$$and$$

$$P ( f \vert {m}_{j}^{N} ) = {\prod }_{h} \frac{B ( {f}_{h} + {α}_{j}^{N} , 1 - {f}_{h} + {β}_{j}^{N} )}{B ( {α}_{j}^{N} , {β}_{j}^{N} )}$$

respectively, for a given fragment f^{c};

αTⱼ, β^{T}ⱼ, α^{N}ⱼ and β^{N}ⱼ are parameters of tumor or normal class beta distributions of MFR on MCB j, which is estimated from m^{T}ⱼ and m^{N}ⱼ;

m^{T}ⱼ is the tumor class methylation pattern on MCBj and m^{N}ⱼ is the normal class methylation pattern on MCBj;

fₕ is 0 or 1;

θ is estimated by a grid search;

and w_{C} is the weight assigned for f^{c}.

10. The method of aspect 9, wherein W_{c} is one of

MR
MR²
\sqrt{MR}
$\frac{1}{log MR}$
$\{ \begin{matrix}1 , & MR \geq {MR}_{b} \vert MR \leq {MR}_{a} \\ 0 , & {MR}_{a} < MR < {MR}_{b}\end{matrix}$

wherein MR is the percentage of methylated CpGs of each fragment, MR_{b} is the threshold of MR for methylated fragments, and MRₐ is the threshold of MR for unmethylated fragments.

11. The method of aspect 9 or 10, wherein the sample is a plasma sample.

12. The method of aspect 11, wherein the DNA is cell-free DNA.

13. A method of treating a subject having cancer, the method comprising:

(A) determining the ctDNA Fraction (CTDF) value of a test subject according to the method of any one of aspects 9-12; and

(B) determining that the test subject has cancer based on the CTDF of (A); and

(C) treating the test subject.

It shall be noted that the preceding are merely examples of embodiments. Other exemplary embodiments are apparent from the entirety of the description herein. It will also be understood by one of ordinary skill in the art that each of these embodiments may be used in various combinations with the other embodiments provided herein.

The following examples further illustrate the invention but, of course, should not be construed as in any way limiting its scope.

### EXAMPLE 1

This Example demonstrates calculation of Methylated Fragment Ratio (MFR) and Unmethylated Fragment Ratio (UFR), in accordance with embodiments of the invention.

### 1.1 Defining Methylation-Correlated Blocks (MCBs)

CpGs meeting the following three criteria are merged into an MCB:

(1) The distance between CpGᵢ and CpGᵢ₊₁ is less than Distanceₘₐₓ, where Distanceₘₐₓ is customized;

(2) The correlation between CpGᵢ and CpGᵢ₊₁ is no less than Correlationₘᵢₙ, where the correlation between CpGs is measured by the Pearson Correlation Coefficient, and Correlationₘᵢₙ is customized; and

(3) The minimum number of CpGs contained in an MCB is no less than cₘᵢₙ, where cₘᵢₙ is customized.

To calculate the correlation between CpGᵢ and CpGᵢ₊₁, beta-values of CpGᵢ and CpGᵢ₊₁ of a group of samples, {sample₁, . . . , sample_{N}}, are first calculated. Specifically, the Person Correlation Coefficient can be calculated by the following formula:

$$C o {r}_{i , i + 1} = \frac{{\sum }_{n = 1}^{N} ( {beta}_{n , i} - {\overline{beta}}_{i} ) ( {beta}_{n , i + 1} - {\overline{beta}}_{i + 1} )}{\sqrt{{\sum }_{n = 1}^{N} {( {beta}_{n , i} - {\overline{beta}}_{i} )}^{2}} \sqrt{{\sum }_{n = 1}^{N} {( {beta}_{n , i + 1} - {\overline{beta}}_{i + 1} )}^{2}}}$$

where Cor_{i,i+1} is the correlation between CpGᵢ and CpGᵢ₊₁, beta_{n,i} and beta_{n,i+1} are the beta-values of sample n on CpGᵢ and CpGᵢ₊₁, b̅e̅t̅a̅ᵢ and b̅e̅t̅a̅ᵢ₊₁ are the mean beta-values among {sample₁, . . . , sample_{N}} on CpGᵢ and CpGᵢ₊₁.

With an increasing cₘᵢₙ, Correlationₘᵢₙ or a decreasing Distanceₘₐₓ, MCBs with less strong correlations are filtered out, which means the signals on the remaining MCBs are more reliable. As number of MCBs becomes smaller, though, information can be lost. Parameters which balance the reliability and the amount of data can be selected.

### 1.2 Combining Read-Pairs Into Fragments

Paired-end sequencing platforms read from both ends of ligated DNA fragments, and produce two reads for each sequence, R1 and R2.

These reads are de-duplicated and filtered according to their mapping qualities (using the program Bowtie2 (Langmead et al., Nature Methods, 9:357-359 (2012), incorporated by reference herein) and conversion rates. The conversion rate is computed using non-CpG Cs covered by the read:

$$Conversion rate = \frac{Number of non - CpG Cs read as T}{Number of non - CpG Cs}$$

If the conversion is successfully done, the conversion rate is 100%, and all of the non-CpG Cs should be read as Ts in the sequencing data.

After filtering reads with mapping qualities less than 20 and conversion rates less than 95%, the remaining read-pairs are merged into a fragment before analysis in order to restore the methylation status of the original DNA fragment comprehensively.

If R1 and R2 overlap and the certain bases are different from each other, bases on the read with a higher average quality score will be selected. If the overlapped bases are different and the average quality scores are equal, the selection will be random between R1 and R2.

### 1.3 Identifying Methylated Fragments of MCB

Methylation statuses of CpGs contained in an MCB are extracted and checked by fragment. For each MCB, fragments covering a minimum of xₘᵢₙ CpGs on the MCB are included.

The joint-methylation-status of H CpGs contained in MCBⱼ of a fragment is denoted as f={f₁, f₂, . . . }, where the binary value fₕ is 0 or 1, representing methylated or unmethylated status of the CpG site h in fragment f. Using the joint-methylation-status, the percentage of methylated CpGs of each fragment is computed as

$$M R = \frac{{\sum }_{h = 1}^{H} {f}_{h}}{H} .$$

Fragments with MR higher than or equal to MR_{b} in MCBⱼ are identified as methylated fragments, while those with MR lower than or equal to MRₐ are identified as unmethylated fragments. The rest are categorized as intermediate fragments.

Parameter xₘᵢₙ should be an integer no larger than cₘᵢₙ, described above in section 1.1. MRₐ and MR_{b} should range from 0 to 1, while MR_{b} should be larger than MRₐ. Parameters can be adjusted according to user preference.

For the Examples, the values are xₘᵢₙ=3, MR_{b}=1, and MRₐ=0.

### 1.4 Calculating MFR and UFR of MCB

Under the criteria of section 1.3, H fragments which cover a specific MCBⱼ are divided into three groups: methylated, unmethylated and intermediate fragments.

Methylated Fragment Ratio (MFR) of MCBⱼ is calculated by

$$M F {R}_{j} = \frac{{{count}^{M}}_{j}}{{{count}^{M}}_{j} + {{count}^{U}}_{j} + {{count}^{I}}_{j}}$$

### and Unmethylated Fragment Ratio (UFR) of MCBⱼ is

$$U F {R}_{j} = \frac{{{count}^{U}}_{j}}{{{count}^{M}}_{j} + {{count}^{U}}_{j} + {{count}^{I}}_{j}}$$

where count^{M}ⱼ=Σₕ₌₁^{H}(MRₕ≥MR_{b}) indicates the number of methylated fragments of MCBⱼ, count^{U}ⱼ=Σₕ₌₁^{H}(MRₕ≤MRₐ) indicates the number of unmethylated fragments of MCBⱼ, count^{U}ⱼ=Σₕ₌₁^{H}(MRₐ<MRₕ<MR_{b}) indicates the number of unmethylated fragments of MCBⱼ.

### EXAMPLE 2

This Example demonstrates the original Methylation Score Model, as described in Liu et al., Ann. Oncol., 29: 1445-1453 (2018), incorporated by reference herein.

### 2.1 Selecting Differential Hypermethylated CpGs

The first step of the Methylation Score Model is to find hypermethylated CpGs, which are defined as CpGs with higher methylation level in the case group than in the control group.

Commonly, moderated t-test is performed by using the “Limma” package from R to compare the methylation level between groups. Beta-values are logit-transformed to M-values before the test:

$${M}_{n , i} = log 2 \frac{b e t {a}_{n , i}}{1 - b e t {a}_{n , i}}$$

where M_{n,i} is the M-value of sampleₙ on CpGᵢ, and beta_{n,i} is the beta-value of sampleₙ on CpGᵢ. pᵢ is the p-value of moderated t-test comparing the mean M-value of CpGᵢ between cases and controls. FDRᵢ, the Benjamini-Hochberg critical value for pᵢ, is then computed to control the false discovery/positive rate (FDR).

To decide whether a CpG is hypermethylated or not, the difference of the mean beta-value between groups is calculated. The difference of CpGᵢ is:

$${diff}_{i} = \overline{{beta}_{i}^{case}} - \overline{{beta}_{i}^{control}}$$

where b̅e̅t̅a̅^̅{̅c̅a̅s̅e̅}̅ᵢ is the mean beta-value of CpGᵢ among case group, while b̅e̅t̅a̅^̅{̅c̅o̅n̅t̅r̅o̅l̅}̅ᵢ is the mean among control group.

If FDRᵢ is smaller than 0.05 and diffᵢ is positive and larger than the pre-defined cutoff diffₘᵢₙ, then CpGᵢ is a differential hypermethylated CpG. It is selected as a marker for building the Methylation Score Model.

### 2.2 Generating Baseline Distributions of Beta-Value

For each selected hypermethylated CpGᵢ, beta-values of control samples are assumed to follow a normal distribution with a mean of μ^{control} ᵢ and a standard deviation of σ^{control}ᵢ:

$$b e t {a}_{n , i} ∼ Norm ( {{μ}^{control}}_{i} , {{σ}^{control}}_{i} )$$

where μ^{control}ᵢ is the mean of beta-value of CpGᵢ among control samples, and σ^{control}ᵢ is the standard deviation of beta-value of CpGᵢ among control samples.

### 2.3 Computing Per-CpG P-Value

With a known baseline distribution Norm(μ^{control}ᵢ, σ^{control}ᵢ) and a known beta-value beta_{n,i}, the Z-score of sampleₙ on CpGᵢ can be computed as

$${Z}_{n , i} = \frac{b e t {a}_{n , i} - {{μ}^{control}}_{i}}{{{σ}^{control}}_{i}} .$$

This Z-score is then transformed to a p-value p_{n,i}.

After repeating this process for N samples and I CpGs, for each sampleₙ, a set of p-values {p_{n,1}, . . . , p_{n,I}} is obtained.

### 2.4 Computing Final Methylation Score

The final score of sampleₙ is a weighted average of the log-transformed p-value from section 2.3:

$$S c o r {e}_{n} = \frac{- 2 {Σ}_{i = 1}^{I} {depth}_{n , i} \cdot ln {p}_{n , i}}{{Σ}_{i = 1}^{I} {depth}_{n , i}}$$

where depth_{n,i} is the sequencing depth of sampleₙ on CpGᵢ.

This score indicates the overall difference of the methylation level between the tested sample and the baseline distribution. A higher score is associated with higher probability of being a cancer case. Cutoff can be set as the 95th percentile/maximum of the control group, or any rational value.

### EXAMPLE 3

This Example demonstrates the Methylation Score Model modified in accordance with embodiments of the invention.

### 3.1 Selecting Differential MCBs

Markers in the modified model are not hypermethylated CpGs but hypermethylated MCBs. A similar selection procedure is performed on J candidate MCBs defined in Example 1, section 1.1.

The methylation level of MCB can either be the mean beta-values of CpGs on MCB or MFR/UFR calculated as in Example 1, section 1.4.

If MFRs are used, moderated t-tests are performed on logit-transformed MFRs to generate FDRs, according to which differential MCBs can be selected. Differences between the mean case MFRs and mean control MFRs are used to determine the direction of differential MCBs.

Logit-transformed MFR of sampleₙ on MCBⱼ:

$${logit MCB}_{n , j} = log 2 \frac{M F {R}_{n , j}}{1 - M F {R}_{n , j}} .$$

FDR of MCBⱼ is FDRⱼ. The difference of MCBⱼ is

$${diff}_{j} = \overline{{{MFR}^{case}}_{j}} - \overline{{{MFR}^{control}}_{j}} .$$

If FDRⱼ is smaller than 0.05 and diffⱼ is positive and larger than the pre-defined cutoff diffₘᵢₙ, MCBⱼ is a differential hypermethylated MCB and is selected as a marker for the modified model. M̅F̅R̅^̅{̅c̅o̅n̅t̅r̅o̅l̅}̅ⱼ and M̅F̅R̅^̅{̅c̅a̅s̅e̅}̅ⱼ are mean MFR of MCBⱼ in control and case groups, respectively.

Although hypermethylated MCBs are selected here, the model is applicable to a global hypomethylated pattern in tumor cells: If the data is generated using a hypomethylation panel, hypomethylated MCBs can also be selected as markers. UFR instead of MFR will be used as the measurement of methylation level.

### 3.2 Generating Baseline Distributions of MFR

The original methylation model assumes normal distributions for beta-values of CpGs, but the natural distributions of beta-value are far from a normal distribution. For the model, the logit transformations of the methylation level measurement can be used.

MFR is used to measure the methylation level of hypermethylated MCB. For each selected hypermethylated MCBⱼ, logit-transformed MFRs of control samples are taken to follow a normal distribution with a mean of μ^{control}ⱼ and a standard deviation of σ^{control}ⱼ:

$$logit M C {B}_{n , j} ∼ Norm ( {μ}_{j}^{control} , {σ}_{j}^{control} )$$

where μ^{control}ⱼ and σ^{control}ⱼ are the mean and the standard deviation of logit-transformed MFR of MCBⱼ among control samples.

If hypomethylated MCBs are used, methylation level of these MCBs will be measured by UFR. The distribution of logit-transformed UFRs among control samples is:

$${logit MCB}_{n , j} ∼ Norm ( {{μ}^{control}}_{j} , {{σ}^{control}}_{j} )$$

where μ^{control}ⱼ and σ^{control}ⱼ are the mean and the standard deviation of logit-transformed UFR of hypomethylated MCBⱼ among control samples.

Although a normal distribution for logit-MFR or logit-UFR is used here, this is not the only option. Other distributions such as beta distribution and Poisson distribution are good substitutes.

### 3.3 Computing Per-MCB P-Value

With a known baseline distribution Norm(μ^{control}ⱼ, σ^{control}ⱼ) and a known MFR_{n,j}, the Z-score of sampleₙ on MCBⱼ can be computed by

$${Z}_{n , j} = \frac{{logit MFR}_{n , j} - {{μ}^{control}}_{j}}{{{σ}^{control}}_{j}} .$$

This Z-score is then transformed to a p-value p_{n,j}.

After repeating this process for N samples and J MCBs, a set of p-value {p_{n,1}, . . . , p_{n,J}} are obtained for each sampleₙ.

If hypomethylated MCBs are used, computation of p-value is almost the same as above, except instead of MFR_{n,j}, UFR_{n,j} is used to calculate the Z-score of sampleₙ on MCBⱼ. Baseline distribution of UFR in hypomethylated MCBⱼ is Norm(μ^{control}ⱼ, σ^{control}ⱼ), and Z-score is computed by

$${Z}_{n , j} = \frac{{logit UFR}_{n , j} - {{μ}^{control}}_{j}}{{{σ}^{control}}_{j}} .$$

The baseline distribution is not necessarily a normal distribution. If any distribution other than normal distribution is used, the p-value calculation will be changed correspondingly.

### 3.4 Computing Final Methylation Score

The final score of sampleₙ is a weighted average of the log-transformed p-value set from section 3.3:

$$S c o r {e}_{n} = \frac{- 2 {Σ}_{j = 1}^{J} {c}_{j} \cdot ln {p}_{n , j}}{{Σ}_{j = 1}^{J} {c}_{j}}$$

where cⱼ is the weight of MCBⱼ, which can optionally be count_{n,j}, the number of fragments on MCBⱼ from sampleₙ.

This score indicates the overall difference of the methylation level between the tested sample and the control group. A higher score is associated with higher probability of being a cancer case. Cutoff can be set as the 95th percentile, the maximum of the control group, or any rational value.

### 3.5 Alternative Weights in Score Calculation

In section 3.4, the final methylation score is a weighted average of the MCB-level p-value. The number of fragments are used as weight, based on the assumption that each fragment contributes equally to the score calculation. Therefore, weights of MCBs with higher coverage are higher.

From another perspective, weights of MCBs can be assigned according to their importance. Since methylation score is interpreted as the overall difference of the methylation level from the control group, importance of an MCB can be equated with the difference between cases and controls. In section 3.1, when selecting differential MCBs, the FDR and the mean methylation level difference between groups for each MCB were computed. Weight of an MCB can either depend on FRD or on mean difference between groups. For example, abs(diff)ⱼ or −ln FDRⱼ for MCBⱼ.

Taking into account both the fragment-level contribution and the MCB-level importance, a combined weight can be used, such as −count_{n,j}·ln FDRⱼ.

### EXAMPLE 4

This Example demonstrates the original CancerDetector Model, as described in Li et al., Nuc. Acids Res., 46: e89 (2018), incorporated by reference herein.

### 4.1 Selecting Frequently Differential Methylation Regions (FDMR)

CpGs are grouped into CpG clusters before marker selection. Two adjacent CpG sites are grouped into a CpG cluster if their flanking regions (100 bp up- and downstream) overlap.

These CpG clusters are candidates for FDMR and are further refined:

(1) At least three CpG sites (in the microarray data) are included in a cluster to obtain a robust measurement of methylation values in the solid tumor samples;

(2) The cluster is reasonably sized; and

(3) As many clusters that span within a type of genomic region (either CpG islands or shores) as possible are kept.

Since CancerDetector is designed for low coverage sequencing data like Whole Genome Bisulfite Sequencing (WGBS) and Reduced Representation Bisulfite Sequencing (RRBS), in order to obtain reliable values, the methylation level is calculated by CpG clusters and is defined as the average methylation level of all CpG sites in the cluster. This means the methylation levels of CpGs in same CpG cluster are even.

FDMRs are selected from CpG clusters, and should meet the following criteria:

(1) Methylation statuses are differential between matched tumor and normal tissues in more than half of the matched pairs; and

(2) The difference between the medians of its methylation levels in two classes is greater than a cutoff.

### 4.2 Building Beta-Value Distributions

Given a region, the methylation levels of all samples in a class are modeled to follow a beta distribution. Distribution of the methylation level on FDMRₖ is Beta(α^{T}ₖ, β^{T}ₖ) in the tumor class and Beta(α^{N}ₖ, β^{T}ₖ) in the normal class. The parameters of a Beta distribution can be determined from the sample population of a class, using either the method of moments or maximum likelihood.

### 4.3 Calculating Per-Read Likelihood

Each cfDNA read is classified as normal class or tumor class based on the joint-methylation-status of multiple CpG sites contained in FDMR on that read. The joint-methylation-status in a cfDNA read is denoted as r={r₁, r₂, . . . }, where the binary value rₕ is 0 or 1, representing methylated or unmethylated status of the CpG site h in read r. The binary vector r is modeled by the Beta-Bernoulli distribution.

Given the tumor class methylation pattern m^{T}ₖ of FDMR k, the tumor class likelihood of read r can be calculated as below:

$$P ( r \vert {{m}^{T}}_{k} ) = {\prod }_{h} P ( {r}_{v} \vert Beta ( {{α}^{T}}_{v} , {{β}^{T}}_{v} ) ) = {\prod }_{h} \frac{B ( {r}_{v} + {{α}^{T}}_{v} , 1 - {r}_{v} + {{β}^{T}}_{v} )}{B ( {{α}^{T}}_{v} , {{β}^{T}}_{v} )}$$

where B(x,y) is the beta function, ν represents CpG site ν in FDMR k, α^{T}_{ν} and β^{T}_{ν} are parameters of the tumor class beta distribution of CpG ν estimated from m^{T}ₖ.

As defined in section 4.1, the methylation level of a FDMR is defined as the average methylation level of all CpG sites in the region. Therefore, the parameter of CpG ν can also be denoted as α^{T}ₖ and β^{T}ₖ. The formula is then translated into:

$$P ( r \vert {{m}^{T}}_{k} ) = {\prod }_{h} \frac{B ( {r}_{k} + {{α}^{T}}_{k} , 1 - {r}_{j} + {{β}^{T}}_{k} )}{B ( {{α}^{T}}_{k} , {{β}^{T}}_{k} )}$$

Similarly, with the normal class methylation pattern m^{N}ₖ, the normal class likelihood of read r is:

$$P ( r \vert {{m}^{N}}_{k} ) = {\prod }_{h} \frac{B ( {r}_{k} + {{α}^{N}}_{k} , 1 - {r}_{j} + {{β}^{N}}_{k} )}{B ( {{α}^{N}}_{k} , {{β}^{N}}_{k} )}$$

where α^{N}ₖ and β^{N}ₖ are parameters of the normal class beta distribution of FDMR k estimated from m^{N}ₖ.

### 4.4 Estimating ctDNA Fraction (CTDF)

Methylation pattern of all K FDMR is denoted as M={(m^{T}ₖ,m^{N}ₖ)}, k=1, . . . , K. The binary vector set of C reads covering FDMRs is denoted as R={r^{c}}. Reads are assumed to be from one of the two classes, the tumor class or the normal class. CTDF θ(0<θ<1) is estimated by maximizing the log-likelihood, which is calculated by the formula:

$$log P ( R \vert θ , M ) = {\sum }_{c} log ( θ \cdot P ( {r}^{c} \vert {m}_{k}^{T} ) + ( 1 - θ ) \cdot P ( {r}^{c} \vert {m}_{k}^{N} ) )$$

where k is the FDMR where r^{c} covers, P(r^{c}|m^{T}ₖ) and P(r^{c}|m^{N}ₖ) are calculated as in section 4.3.

Estimation of θ is done by a grid search. One thousand one fraction values uniformly distributed between 0% and 100% are exhaustively enumerated to find the global optimization.

### EXAMPLE 5

This Example demonstrates the CancerDetector Model modified in accordance with embodiments of the invention.

### 5.1 Selecting Differential MCBs

Markers in the modified model are MCBs, not FDMR. The procedure is the same as in Example 3, section 3.1.

### 5.2 Building MFR Distributions

MFR distributions are built in the modified model. MFRs can be modeled by beta distributions.

Distribution of MFR on MCBⱼ is Beta(α^{T}ⱼ, β^{T}ⱼ) in tumor class and Beta(α^{N}ⱼ, β^{N}ⱼ) in normal class. The parameters are determined from the tumor class samples and from the normal class samples, respectively.

### 5.3 Calculating Per-Fragment Likelihood

Similar as the Modified Methylation Score Model, paired reads are firstly merged into fragments, according to the protocol in Example 1, section 1.2. This means that the likelihood is now calculated by fragment, not by read.

The joint-methylation-status in a cfDNA fragment is denoted as f=f{f₁, f₂, . . . }, where the binary value fₕ is 0 or 1, representing methylated or unmethylated status of the CpG site h in fragment f. The binary vector f is modeled by the Beta-Bernoulli distribution.

Given the tumor and normal class methylation pattern m^{T}ⱼ and m^{N}ⱼ on MCB j, the tumor and normal class likelihoods of fragment f are:

$$P ( f \vert {m}_{j}^{T} ) = {\prod }_{h} \frac{B ( {f}_{h} + {α}_{j}^{T} , 1 - {f}_{h} + {β}_{j}^{T} )}{B ( {α}_{j}^{T} , {β}_{j}^{T} )} and$$

$$P ( f \vert {m}_{j}^{N} ) = {\prod }_{h} \frac{B ( {f}_{h} + {α}_{j}^{N} , 1 - {f}_{h} + {β}_{j}^{N} )}{B ( {α}_{j}^{N} , {β}_{j}^{N} )}$$

where α^{T}ⱼ, β^{T}ⱼ, α^{N}ⱼ and β^{N}ⱼ are parameters of tumor or normal class beta distributions of MFR on MCB j, which is estimated from m^{T}ⱼ and m^{N}ⱼ.

### 5.4 ctDNA Fraction (CTDF)

Methylation pattern of all J MCBs is denoted as M={(m^{T}ⱼ,m^{N}ⱼ)}, j=1, . . . , J. The binary vector set of C fragments covering MCBs is denoted as F={f^{c}}.

Similar to the original CancerDetector Model, CTDF θ(0<θ<1) can be estimated by a maximum likelihood estimation (MLE) method by maximizing the global log-likelihood, but here weights are added as the coefficients of per-fragment log-likelihood. The weighted log-likelihood is calculated by the following formula:

$$log P ( F \vert θ , M ) = {\sum }_{c} {w}_{c} \cdot log ( θ \cdot P ( {f}^{c} \vert {m}_{j}^{T} ) + ( 1 - θ ) \cdot P ( {f}^{c} \vert {m}_{j}^{N} ) )$$

where j is the MCB covered by f^{c}, P(f^{c}|m^{T}ⱼ) and P(f^{c}|m^{N}ⱼ) are calculated in section 5.3, w_{c} is the weight assigned for f^{c}.

To reduce the noise caused by technical artifacts, weight of an intermediate fragment is set to a lower value than a fully methylated/unmethylated fragment. Here are some examples of weights that can be used:

| | Weight |
|---|---|
| A | MR |
| B | MR² |
| C | \sqrt{MR} |
| D | $\frac{1}{log MR}$ |
| E | $\{ \begin{matrix}1 , & MR \geq {MR}_{b} \vert MR \leq {MR}_{a} \\ 0 , & {MR}_{a} < MR < {MR}_{b}\end{matrix}$ |

where MR is the percentage of methylated CpGs on MCB of a fragment (MR is defined in Example 1, section 1.3).

Weight E is applied in the dataset, MRₐ=0 and MR_{b}=1 as Example 1, section 1.3, because it can improve the model performance. The following values can be set: w=1 for fully methylated/unmethylated fragments, and w=0 for intermediate methylated fragments.

Estimation of θ is done by grid search. Ten fraction values uniformly distributed between 0% and 0.1% plus 1000 fraction values uniformly distributed between 0.1% and 100% are exhaustively enumerated to find the global optimization.

### 5.5 Alternative Weights in CTDF Estimation

In section 5.4, weights are assigned to fragments depending on their MRs. These weights are to reduce technical artifacts. Beside technical artifacts, the more CpGs a fragment covers, the more information it provides. As in Example 3, section 3.5, statistical values of MCBs, such as FDR and mean difference, reflect the methylation level difference between groups, implying the importance of MCBs.

Therefore, an example of the updated weight E in section 5.4 is:

$$\{ \begin{matrix}- H \cdot ln F D {R}_{j} , & M R \geq M {R}_{b} \vert M R \leq M {R}_{a} \\ 0 , & M {R}_{a} < M R < M {R}_{b}\end{matrix}$$

where H is the number of CpGs in MCBⱼ covered by the fragment, FDRⱼ is the FDR computed for MCBⱼ.

In this case, intermediate methylated fragments are not used, weights of methylated and unmethylated fragments are associated with the importance of the MCB (defined as the significance of difference between groups) and the number of covered CpGs in that MCB.

### EXAMPLE 6

This Example demonstrates use of the inventive methods, in accordance with embodiments of the invention.

Four samples of plasma from lung cancer patients with high CTDF were diluted at a rate of 1:27, 1:81, and 1:243 respectively. The samples underwent target sequencing by enzyme-based conversion. Additionally, to build the models, DNA extracted from 50 healthy plasma samples and 195 Formalin-fixed Paraffin-embedded (FFPE) tissues were converted and sequenced using the same panel. Among the 195 FFPE samples, 11 were from lung tumors.

Correlations of methylation level between adjacent CpGs were measured using the beta-values of the 195 FFPE samples. MCBs were defined as in Example 1, section 1.1, setting Distanceₘₐₓ=100 bp, Correlationₘᵢₙ=0.95 and cₘᵢₙ=3. MFR of the MCBs of the diluted plasmas and healthy plasmas were calculated as in Example 1, section 1.2-1.4, setting xₘᵢₙ=3, MR_{b}=1, and MRₐ=0. Strategy for MCB marker selection was described in Example 3, section 3.1. The mean beta-values of each MCB between the 11 lung cancer tissues and the 50 healthy plasmas were compared and only hypermethylated MCBs with FDR less than 0.05 and diffₘᵢₙ larger than 0.1 were kept. Furthermore, to ensure that the methylation level was low in the healthy plasma, mean beta-values less than 0.02 in healthy plasmas was required. 208 MCBs met the criterion were selected as markers.

Fifty healthy plasma samples were used to build the baseline in the Methylation Score Model and the normal class baseline in the CancerDetector Model. Eleven lung cancer tissues were used to build the tumor class baseline in the CancerDetector Model.

To verify the superiority of MFR, the performance between Model A (TABLE 1) and B (TABLE 2) was compared: Model A—modified Methylation Score Model using MFR as the measure of methylation level and the number of fragments as the weight, Model B—modified Methylation Score Model using the mean beta-value of CpGs on an MCB as the measure of methylation level and the mean depth as the weight.

The performance between Model C (TABLE 3) and Model D (TABLE 4) was compared: Model C—modified CancerDetector Model with weight

$$\{ \begin{matrix}1 , & M R \geq M {R}_{b} \vert M R \leq M {R}_{a} \\ 0 , & M {R}_{a} < M R < M {R}_{b}\end{matrix} .$$

The parameters of baseline distributions of an MCB were tuned using the mean beta-value of CpGs on the MCB. Model D—modified CancerDetector Model without assigning weights. The parameters of baseline distributions of an MCB were tuned using the mean beta-value of CpGs on the MCB. Using the highest predicted value in the healthy individuals as the cutoff, samples with a value exceeding the cutoff were predicted as cancer cases and are bold and italicized with an asterisk in the tables below.

TABLE 1
Methylation Score Model (Original)

| | 1/27 | 1/81 | 1/243 |
|---|---|---|---|
| Person 1 | 1.96 | 1.45 | 1.23 |
| Person 2 | [symbol] | 2.02 | 1.12 |
| Person 3 | 1.18 | 0.83 | 0.68 |
| Person 4 | 3.62 | 1.41 | 0.94 |

TABLE 2
Methylation Score Model (Modified - MFR)

| | 1/27 | 1/81 | 1/243 |
|---|---|---|---|
| Person 1 | [symbol] | 2.86 | 1.48 |
| Person 2 | [symbol] | [symbol] | [symbol] |
| Person 3 | [symbol] | 2.13 | 2.01 |
| Person 4 | [symbol] | [symbol] | 2.82 |

TABLE 3
CancerDetector Model (Original)

| | 1/27 | 1/81 | 1/243 |
|---|---|---|---|
| Person 1 | 1.60% | 1.00% | 0.79% |
| Person 2 | 3.40% | 1.70% | 1.00% |
| Person 3 | 1.20% | 1.00% | 0.83% |
| Person 4 | 2.20% | 1.10% | 0.85% |

TABLE 4
CancerDetector Model (Modified)

| | 1/27 | 1/81 | 1/243 |
|---|---|---|---|
| Person 1 | [symbol] | 0.18% | 0.09% |
| Person 2 | [symbol] | [symbol] | 0.18% |
| Person 3 | 0.21% | 0.12% | 0.10% |
| Person 4 | [symbol] | [symbol] | 0.16% |

It was found that the highest Methylation Score in healthy individuals was reduced from 6.92 to 4.36, the highest CTDF of healthy individuals estimated by CancerDetector reduced from 6.5% to 0.32%, and the sensitivity of both models were greatly improved.

### EXAMPLE 7

This Example demonstrates use of the inventive methods, in accordance with embodiments of the invention.

Four samples of plasma from cancer patients with high CTDF were diluted at a rate of 1:1, 1:3, 1:9, 1:27, 1:81, and 1:243 respectively. The samples underwent target sequencing by bisulfite conversion. Additionally, to build the models, DNA extracted from 41 healthy plasmas, 35 lung cancer plasmas and 59 FFPE lung tissues were converted and sequenced using the same panel.

Methylation level between adjacent CpGs were measured using the beta-values of the 59 FFPE samples. MCBs were defined as in Example 1, section 1.1, setting Distanceₘₐₓ=100 bp, Correlationₘᵢₙ=0.95 and cₘᵢₙ=3. MFR of the MCBs of the diluted plasmas and healthy plasmas were calculated as in Example 1, section 1.2-1.4, setting xₘᵢₙ=3, MR_{b}=1, and MRₐ=0.

Strategy for MCB marker selection is as described in Example 3, section 3.1. The mean beta-values of each MCB between the 35 lung cancer plasmas and the 41 healthy plasmas were compared and only hypermethylated MCBs with FDR less than 0.05 and diffₘᵢₙ larger than 0.1 were kept. Furthermore, to ensure that the methylation level was low in the healthy plasma, the mean beta-values of MCB was required to be less than 0.02 in over 80% healthy plasmas. Twenty-one MCBs met the criterion were selected as markers.

Forty-one healthy plasmas were used to build the baseline in the Methylation Score Model and the normal class baseline in the CancerDetector Model. Thirty-five lung cancer plasmas were used to build the tumor class baseline in the CancerDetector Model.

The performance between Model A (TABLE 5) and B (TABLE 6) was compared: Model A—modified Methylation Score Model using MFR as the measure of methylation level and the number of fragments as the weight, Model B—modified Methylation Score Model using the mean beta-value of CpGs on an MCB as the measure of methylation level and the mean depth as the weight.

The performance between Model C (TABLE 7) and Model D (TABLE 8) was compared: Model C—modified CancerDetector Model with weight

$$\{ \begin{matrix}1 , MR \geq {MR}_{b} \vert MR \leq {MR}_{a} \\ 0 , {MR}_{a} < MR < {MR}_{b}\end{matrix} .$$

The parameters of baseline distributions of an MCB were tuned using the mean beta-value of CpGs on the MCB, Model D—modified CancerDetector Model without assigning weights. The parameters of baseline distributions of an MCB were tuned using the mean beta-value of CpGs on the MCB.

Using the highest predict value in the healthy individuals as the cutoff, samples with a value exceeding the cutoff were predicted as cases and are bold and italicized with an asterisk in the tables below.

TABLE 5
Methylation Score Model (Original)

| | 1/1 | 1/3 | 1/9 | 1/27 | 1/81 | 1/243 |
|---|---|---|---|---|---|---|
| Person 1 | [symbol] | [symbol] | [symbol] | [symbol] | 4.65 | 3.80 |
| Person 2 | [symbol] | [symbol] | [symbol] | [symbol] | [symbol] | 3.84 |
| Person 3 | [symbol] | [symbol] | 5.63 | 4.31 | 3.54 | 3.57 |
| Person 4 | [symbol] | [symbol] | [symbol] | 5.90 | 5.22 | 4.16 |

TABLE 6
Methylation Score Model (Modified - MFR)

| | 1/1 | 1/3 | 1/9 | 1/27 | 1/81 | 1/243 |
|---|---|---|---|---|---|---|
| Person 1 | [symbol] | [symbol] | [symbol] | [symbol] | [symbol] | 2.09 |
| Person 2 | [symbol] | [symbol] | [symbol] | [symbol] | [symbol] | 4.62 |
| Person 3 | [symbol] | [symbol] | 6.35 | 2.56 | 1.20 | 2.86 |
| Person 4 | [symbol] | [symbol] | [symbol] | [symbol] | [symbol] | 0.94 |

TABLE 7
CancerDetector Model (Original)

| | 1/1 | 1/3 | 1/9 | 1/27 | 1/81 | 1/243 |
|---|---|---|---|---|---|---|
| Person 1 | [symbol] | [symbol] | [symbol] | 3.80% | 1.80% | 1.30% |
| Person 2 | [symbol] | [symbol] | [symbol] | 4.90% | 4.30% | 1.20% |
| Person 3 | [symbol] | [symbol] | 2.80% | 1.00% | 1.30% | 1.50% |
| Person 4 | [symbol] | [symbol] | [symbol] | 3.20% | 1.90% | 0.50% |

TABLE 8
CancerDetector Model (Modified)

| | 1/1 | 1/3 | 1/9 | 1/27 | 1/81 | 1/243 |
|---|---|---|---|---|---|---|
| Person 1 | [symbol] | [symbol] | [symbol] | [symbol] | 0.55% | 0.08% |
| Person 2 | [symbol] | [symbol] | [symbol] | [symbol] | [symbol] | 0.32% |
| Person 3 | [symbol] | [symbol] | 0.76% | 0.33% | 0.11% | 0.14% |
| Person 4 | [symbol] | [symbol] | [symbol] | [symbol] | 0.55% | 0.01% |

It was found that the highest Methylation Score in healthy individuals changed from 6.3 to 6.46, which was possibly due to the small-size marker-set; the highest CTDF of healthy individuals estimated by CancerDetector reduced from 4.9% to 0.8%; and sensitivity of both models were improved, although the cutoff of the Methylation Score Model increased.

### EXAMPLE 8

This Example demonstrates use of the inventive methods, in accordance with embodiments of the invention.

Samples compared were RRBS plasma samples from a published paper (Guo et al. Nature Genetics, 49:635-642 (2017), incorporated by reference herein). Different from target sequencing, Reduced Representation Bisulfite Sequencing (RRBS) generates sequencing data which covers a broader region with lower depth (10-20x in this dataset).

RRBS data of 30 healthy, 20 cancer plasmas (10 of lung and colon cancer respectively) and 10 FFPE tissues (5 of lung and colon respectively) were downloaded to build and test the model.

Correlations of methylation level between adjacent CpGs were measured using the beta-values of 10 FFPE samples. MCBs were defined as in Example 1, section 1.1, setting Distanceₘₐₓ=100 bp, Correlationₘᵢₙ=0.7 and cₘᵢₙ=3. MFR of the MCBs of the diluted plasmas and healthy plasmas were calculated as in Example 1, section 1.2-1.4, setting xₘᵢₙ=3, MR_{b}=1, and MRₐ=0.

Strategy for MCB marker selection is as described in Example 3, section 3.1. For each cancer type, the mean beta-values between the 5 tumor tissues and 15 of the 30 healthy plasmas were compared and those kept were only MCBs with FDR less than 0.05, diffₘᵢₙ larger than 0.4 and with average MCB mean beta-value less than 0.05 in healthy plasmas. Fifty-four and 70 MCBs met the criteria and were selected as colon cancer and lung cancer markers respectively.

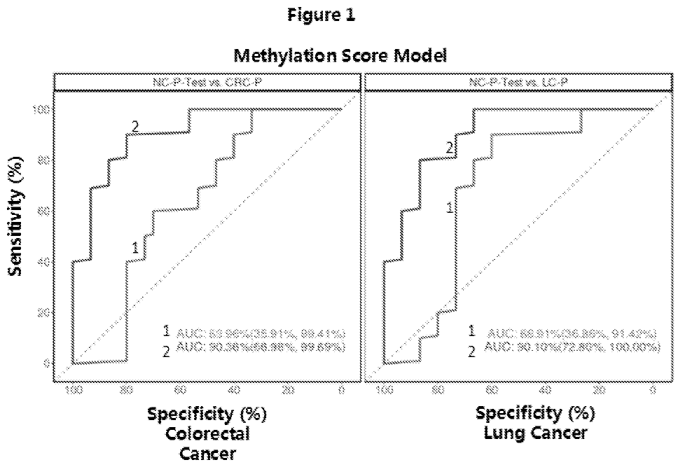

The performance between Model A and B (FIG. 1) was compared: Model A—modified Methylation Score Model using MFR as the measure of methylation level and the number of fragments as the weight, Model B—modified Methylation Score Model using the mean beta-value of CpGs on an MCB as the measure of methylation level and the mean depth as the weight.

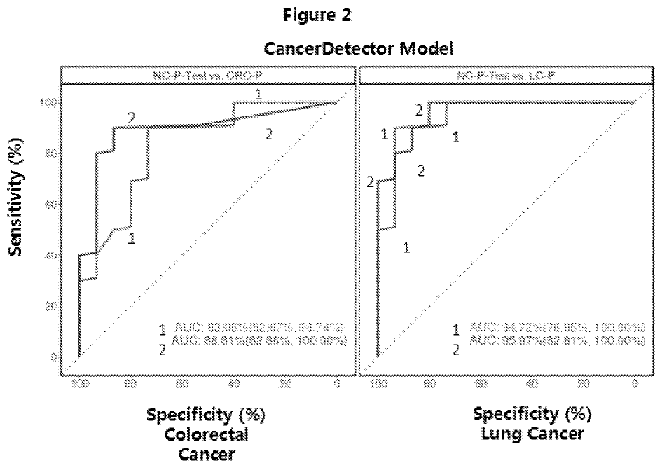

The performance between Model C and Model D (FIG. 2) was compared:

$$Model C \text{-} modified CancerDetector Model with weight \{ \begin{matrix}1 , & M R \geq M {R}_{b} \vert M R \leq M {R}_{a} \\ 0 , & M {R}_{a} < M R < M {R}_{b}\end{matrix} .$$

The parameters of baseline distributions of an MCB were tuned using the mean beta-value of CpGs on the MCB, Model D—modified CancerDetector Model without assigning weights. The parameters of baseline distributions of an MCB were tuned using the mean beta-value of CpGs on the MCB.

It was found that AUC of Methylation Model was improved from less than 70% to 90%, and AUC of CancerDetector Model was improved as well.

Even though RRBS data differs greatly from target sequencing data, the inventive methods worked effectively.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for determining a methylation score of DNA, the method comprising:
(a) providing a sample from a subject;
(b) isolating DNA from the sample of (a);
(c) treating isolated DNA of (b) with bisulfite or enzyme to perform conversion of unmethylated cytosines in the DNA;
(d) performing library construction of the converted DNA of (c) by paired end next generation sequencing (NGS);
(e) obtaining sequencing data from the paired end NGS of (d) and determining DNA sequences of DNA fragments present,
wherein the sequence of a DNA fragment is determined by merging the sequences of read-pairs for the DNA fragment;
(f) identifying methylation status of DNA fragments of (e) by comparing a reference genome to the sequencing data of (e) to determine if a cytosine base in a CpG site within a DNA fragment is methylated or unmethylated;
(g) calculating for Methylation-Correlated Blocks (MCBs) a Methylated Fragment Ratio (MFR) value or an Unmethylated Fragment Ratio (UFR) value or both a MFR value and an UFR value, wherein the MCBs are based on CpGs pre-determined within the DNA; and
(h) calculating a p-value for each of selected differential MCBs,
wherein the selected differential MCBs are selected based on pre-determined MFR and UFR values, and
wherein the p-value is based on a pre-determined baseline distribution of MFR values if selected differential MCBs are hypermethylated or UFR values if selected differential MCBs are hypomethylated; and
(i) calculating a methylation score using the equation
$$S c o r {e}_{n} = \frac{- 2 {\sum }_{j = 1}^{J} {c}_{j} \cdot ln {p}_{n , j}}{{\sum }_{j = 1}^{J} {c}_{j}}$$
wherein cⱼ is the weight of MCBⱼ within sampleₙ, p_{n,j} is the p-value of (h) for MCBⱼ in sampleₙ,
wherein sampleₙ, has J number of MCB, and
wherein the methylation score is a hypermethylation score if selected differential MCBs in (h) are hypermethylated and is a hypomethylation score if selected differential MCBs in (h) are hypomethylated and is a hybrid methylation score if selected differential MCBs in (h) comprise both hypermethylated and hypomethylated MCBs.

2. The method of claim 1, wherein selected differential MCBs in (h) are hypermethylated and wherein a hypermethylation score is calculated in (i).

3. The method of claim 1, wherein selected differential MCBs in (h) are hypomethylated wherein a hypomethylation score is calculated in (i).

4. The method of claim 1, wherein selected differential MCBs in (h) comprise both hypermethylated and hypomethylated MCBs and wherein a hybrid methylation score is calculated in (i).

5. The method of claim 1, wherein the cⱼ is equal to
count_{n,j}, or
−count_{n,j}·ln FDRⱼ,
wherein count_{n,j} is the number of fragments from sampleₙ on MCBⱼ, and FDRⱼ is false discovery/positive rate of MCBⱼ.

6. The method of claim 1, wherein the sample is a plasma sample.

7. The method of claim 6, wherein the DNA is cell-free DNA.

8. A method of treating a subject having cancer, the method comprising:
(A) determining the methylation score of DNA of a test subject according to the method of claim 1; and
(B) determining that the test subject has cancer based on the methylation score of (A); and
(C) treating the test subject.

9. A method for determining a ctDNA Fraction (CTDF) value, the method comprising:
(a) providing a sample from a subject;
(b) isolating DNA from the sample of (a);
(c) treating isolated DNA of (b) with bisulfite or enzyme to perform conversion of unmethylated cytosines in the DNA;
(d) performing library construction of the converted DNA of (c) by paired end next generation sequencing (NGS);
(e) obtaining sequencing data from the paired end NGS of (d) and determining DNA sequences of DNA fragments present,
wherein the sequence of a DNA fragment is determined by merging the sequences of read-pairs for the DNA fragment;
(f) identifying methylation status of DNA fragments of (e) by comparing a reference genome to the sequencing data of (e) to determine if a cytosine base in a CpG site within a DNA fragment is methylated or unmethylated;
(g) calculating for Methylation-Correlated Blocks (MCBs) a Methylated Fragment Ratio (MFR) value, wherein the MCBs are based on CpGs pre-determined within the DNA; and
(h) calculating tumor and non-tumor likelihood values for each of selected differential MCBs,
wherein the selected differential MCBs are selected based on pre-determined MFR values, and
wherein the tumor and non-tumor likelihood values are based on a pre-determined beta distribution of MFR values calculated in (g); and
(i) calculating a ctDNA Fraction (CTDF) value based on the tumor and non-tumor likelihood values determined in (h) using the equation
$$log P ( F \vert θ , M ) = {\sum }_{c} {w}_{c} \cdot log ( θ \cdot P ( {f}^{c} \vert {m}_{j}^{T} ) + ( 1 - θ ) \cdot P ( {f}^{c} \vert {m}_{j}^{N} ) )$$
wherein
j is the MCB covered by f^{c};
P(f^{c}|m^{T}ⱼ) and P(f^{c}|m^{N}ⱼ) are
$$P ( f \vert {m}_{j}^{T} ) = {\prod }_{h} \frac{B ( {f}_{h} + {α}_{j}^{T} , 1 - {f}_{h} + {β}_{j}^{T} )}{B ( {α}_{j}^{T} , {β}_{j}^{T} )} and$$
$$P ( f \vert {m}_{j}^{N} ) = {\prod }_{h} \frac{B ( {f}_{h} + {α}_{j}^{N} , 1 - {f}_{h} + {β}_{j}^{N} )}{B ( {α}_{j}^{N} , {β}_{j}^{N} )}$$
respectively, for a given fragment f^{c};
α^{T}ⱼ, β^{T}ⱼ, α^{N}ⱼ and β^{N}ⱼ are parameters of tumor or normal class beta distributions of MFR on MCB j, which is estimated from m^{T}ⱼ and m^{N}ⱼ;
m^{T}ⱼ is the tumor class methylation pattern on MCBj and m^{N}ⱼ is the normal class methylation pattern on MCBj;
fₕ is 0 or 1;
θ is estimated by a grid search;
and w_{c} is the weight assigned for f^{c}.

10. The method of claim 9, wherein w_{c} is one of
MR
MR²
\sqrt{MR}
$\frac{1}{log MR}$
$\{ \begin{matrix}1 , & MR \geq {MR}_{b} \vert MR \leq {MR}_{a} \\ 0 , & {MR}_{a} < MR < {MR}_{b}\end{matrix}$
wherein MR is the percentage of methylated CpGs of each fragment, MR_{b} is the threshold of MR for methylated fragments, and MRₐ is the threshold of MR for unmethylated fragments.

11. The method of claim 9, wherein the sample is a plasma sample.

12. The method of claim 11, wherein the DNA is cell-free DNA.

13. A method of treating a subject having cancer, the method comprising:
(A) determining the ctDNA Fraction (CTDF) value of a test subject according to the method of claim 9; and
(B) determining that the test subject has cancer based on the CTDF of (A); and
(C) treating the test subject.
